(19) United States
(12) Patent Application Publication
(10) Pub. No.: US 2007/0299310 A1
(43) Pub. Date: Dec. 27, 2007

(54) Device for shielding the lens of a flexible or rigid surgical endoscope

(76) Inventor: Edward H. Phillips, Beverly Hills, CA (US)

Correspondence Address: Frederick Gotha, Suite 823, 80 South Lake Avenue, Pasadena, CA, 91101, US

(21) Appl. No.: 11/471,883

(22) Filed: Jun. 21, 2006

(51) Int. Cl. A61B 1/04

(52) U.S. Cl. 600/127

(57) ABSTRACT

This invention relates to a shield member for shielding the lens of a flexible or rigid surgical endoscope from body fluid, tissue debris, and condensation deposited on a working port valve as the lens passes through the working port lumen in a posterior direction to gain access to the intra-abdominal or pleural cavity regions. The shield member has a shield lumen and is telescopically and slidably carried within the lumen. The shield member is so dimensioned and proportioned such that the distal end of the shield member may be selectively positioned posteriorly of the working port valve thereby permitting the lens to advance axially within the shield lumen and by-pass the working port valve without coming in physical contact with the working port valve.

### FIELD OF THE INVENTION

This invention relates to a device to shield the lens of a flexible or rigid surgical endoscope and rigid laparoscope during posterior passage of the lens within the working port lumen.

### BACKGROUND OF THE INVENTION

Laparoscopic or thoroscopic surgery performed endoscopically requires the placement of working ports in the abdominal or thoracic regions in order to gain access into the intra-abdominal region or pleural cavity. Each working port has an axially extending lumen and an anterior opening and a posterior opening, both of these openings are in communication with the working port lumen. Working ports provide the conduits or lumens through which surgical instruments and flexible or rigid surgical endoscopes enter the operative region.

To position a working port during a surgical procedure, a tiny trocar incision is made in the skin by the surgeon to facilitate the entry of the trocar through, for example, the abdominal wall; the trocar and working port assembly are so placed in the incision so as to permit the trocar, which is mounted in telescopic relationship within the working port, to cut through the abdominal wall. After passing through the wall, the trocar is retracted through the lumen and removed from the working port. Working ports have a valve or a plurality of valves positioned within the working port lumen intermediate the anterior and posterior ends of the working port to provide a seal against the loss of pneumoperitoneum and to assure relatively constant insufflation. With the retraction and removal of the trocar from the working port, the working port lumen acts as a conduit for the video camera lens to gain access to the operative region and for the camera to transmit images of the operative area to the surgeon. However, in retracting and removing the trocar from the working port, body fluids and tissue debris along with condensation are transferred to the working port valve. When the flexible or rigid surgical endoscope is passed posteriorly through the working port valve, the lens of the flexible or rigid surgical endoscope comes in contact with these deposits resulting in a transfer of these deposits to the lens and the images transmitted by the lens to a viewing monitor are consequently blurred. It would therefore be desirable to shield the lens as at it passes posteriorly through the working port lumen and past the working port valve or valves. The present invention is directed to a shield device carried in telescopic relationship with the working port where the shield device is telescopically carried within the working port lumen and has a shield lumen through which the lens passes without coming into physical contact with the working port valve or valves.

### SUMMARY OF THE INVENTION

There is, therefore, provided according to the present invention a device for shielding the lens of a flexible or rigid surgical endoscope or laparoscope during passage of the lens through lumen of the working port after the working port is positioned in the abdominal or thoracic region during an endoscopic surgery procedure.

The present invention is directed toward a shield member which has a distal end, a proximal end, an axis of elongation, and an internal void or shield lumen extending axially through the shield member. In the preferred embodiment, the shield member has a cylindrical shape with a cylindrically shaped shield lumen extending axially therethrough. The shield member has a first opening at its distal end which communicates with the shield lumen and a second opening at its proximate end that communicates with the shield lumen. At its proximate end, the shield member has a radially extending flange or lip for precluding the axially advance of the shield member within the working port lumen upon engagement of the flange or lip with an anterior barrier located adjacent the anterior opening of the working port. The shield lumen is so dimensioned and proportioned to permit the lens of the flexible or rigid surgical endoscope to advance axially through the shield lumen and the shield member is so dimensioned and proportioned such that the shield member may be slidably and telescopically carried by the working port for axial displacement within the working port lumen. This permits the distal end of the shield member to be selectively positioned posteriorly of the working port valve thereby shielding the lens of the flexible or rigid surgical endoscope as the lens advances axially within the shield lumen and past the working port valve or valves.

In another embodiment, the shield member may be made of an elastic material and contain an axially extending slit that communicates with the shield lumen. The elastic material is sufficiently elastic such that the circumferential arcuate dimension of the slit is selectively expandable so as to permit the shield member to be laterally separable from the flexible or rigid surgical endoscope.

### BRIEF DESCRIPTION OF THE DRAWINGS

These and other features and advantages will become appreciated as the same become better understood with reference to the following specification, claims and drawings wherein:

FIG. 1 is a perspective view of a trocar and working port in an assembled configuration.

FIG. 2 is a perspective and expanded view of a working port and trocar with the trocar separated anteriorly of the working port.

FIG. 3 is an illustration in perspective and expanded view of a working port, the shield member of this invention, and the lens portion of a flexible or rigid surgical endoscope.

FIG. 4 illustrates in perspective view the assembly of the shield member of this invention and the working port before insertion of the lens portion of a flexible or rigid surgical endoscope into the shield lumen.

FIG. 5 illustrates in perspective the passage of the lens portion of a flexible or rigid surgical endoscope through the shield lumen of the shield member and past the working port valve.

FIG. 6 illustrates in perspective the retraction of the shield member through the working port lumen and separation of the shield member from the distal flexible or rigid surgical endoscope portion containing the lens.

### DETAILED DESCRIPTION

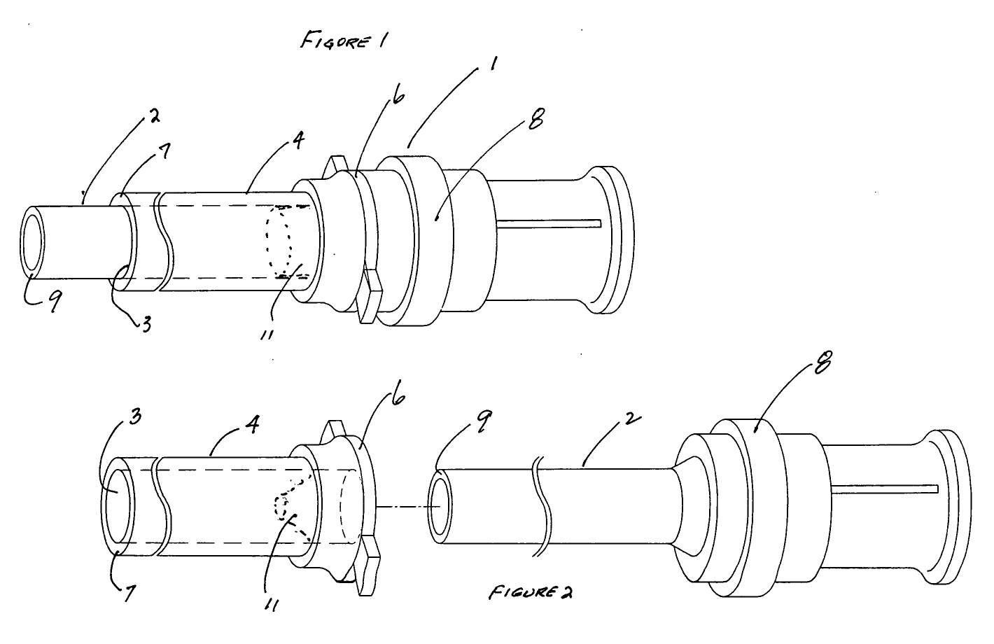

FIG. 1 is an illustration in perspective of a typical trocar and working port assembly 1 of the prior art. In laparoscopic surgery, access to the abdominal cavity is generally achieved by using a trocar 2 that is carried within the lumen 3 of a cannula or working port 4. The trocar 2, or obturator, is used to pierce the abdominal wall (not shown) and is then removed from working port 4 which allows access to the abdominal cavity by a camera system through working port lumen 3. As can be seen in FIGS. 1 and 2, working port 4 has an anterior portion 6 and a posterior end 7. In this description, the anterior portion 6 of working port 4, although the abdomen is not shown, remains anterior of the abdomen after trocar 2 has pierced the abdominal wall and been withdrawn from working port 4. Trocar 2 has an anterior portion 8 and posterior end 9. Typically, although not shown, trocar posterior end 9 contains the trocar cutting blade which pierces the abdominal wall and allows working port 4 to pass through the wall where its lumen 3 communicates with the internal cavity of the abdomen.

Referring again to FIGS. 1 and 2, working port 4 has a working port valve 11 which as can be seen expands to form a seal surrounding the trocar posterior portion as it extends through working port lumen 3. The working port valve 11 acts to prevent loss of pneumoperitoneum and assures relatively constant insufflation. Such valves are commonly used in working ports of the prior art. When the trocar 2 pierces the abdominal wall, body fluids and tissue debris are carried by the posterior portion 9 of the trocar as it is removed form working port 4 where the fluids and debris are transferred to working port valve 11 as the trocar 2 is withdrawn. In addition, the temperature gradient across the valve after the trocar is withdrawn results in condensation forming on the valve. Consequently, when a flexible or rigid surgical endoscope 12 containing a lens 13 passes working port valve 11 in a posterior direction while gaining access to the abdominal cavity, the body fluid, debris, and condensation (not shown) smear lens 13 and blur the visual images transmitted to the monitor screen (not shown). The present invention is directed toward shielding the lens as it passes posteriorly through valve 11.

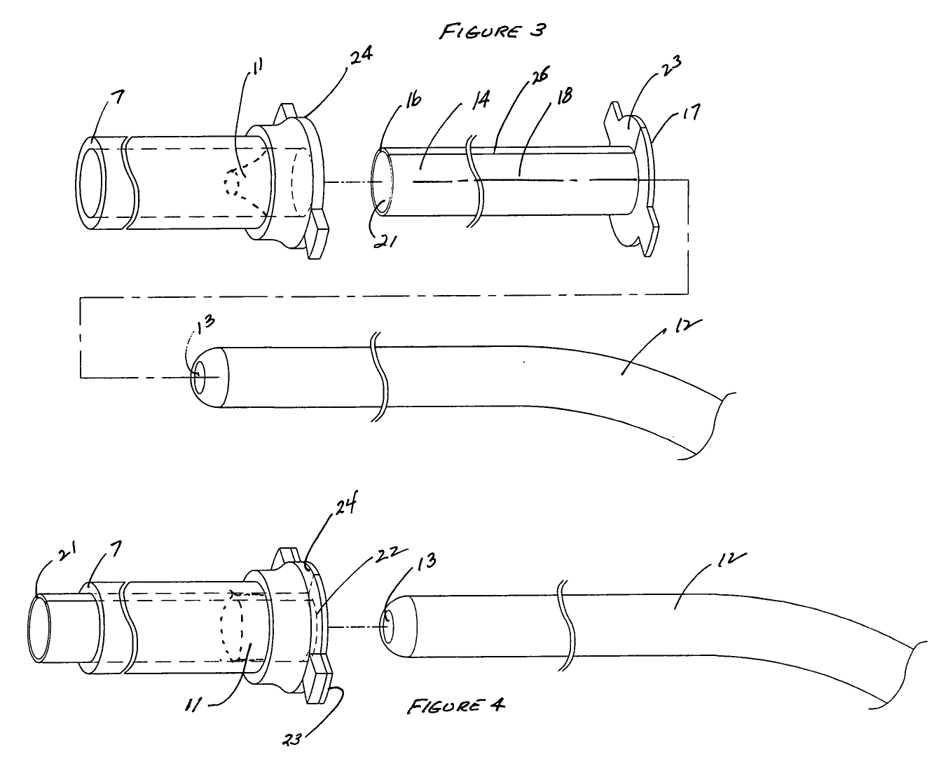

Referring to FIG. 3, a shield member 14 adapted for insertion into working port lumen 3 has a distal end 16 and a proximate end 17, an axis of elongation 18, and an axially extending shield lumen 19. Shield member 14 further has a first opening 21 at its distal end 16 that communicates with shield lumen 19 and a second opening 22 at proximate end 17 (shown in FIG. 4) that communicates with shield lumen 19 forming a cylindrical passageway through the shield.

As is shown in FIG. 3, in one embodiment, shield member 14 has a radially extending flange or lip 23 adjacent proximate end 17. By referring to FIG. 4, it can be seen that shield member 14 is so dimensioned and proportioned that it can be inserted into working port lumen 3 telescopically and extend axially in a posterior direction beyond posterior end 9 of working port 4. The anterior portion 6 of working port 4 has an anterior barrier 24 that limits the posterior travel of shield member 14 through working port lumen 3. Although a radially extending flange from shield member 14 is shown as an embodiment, other methods may be used to limit the axial advance of the shield member through working port lumen 3 to achieve the function of providing a shielded passageway for lens 13.

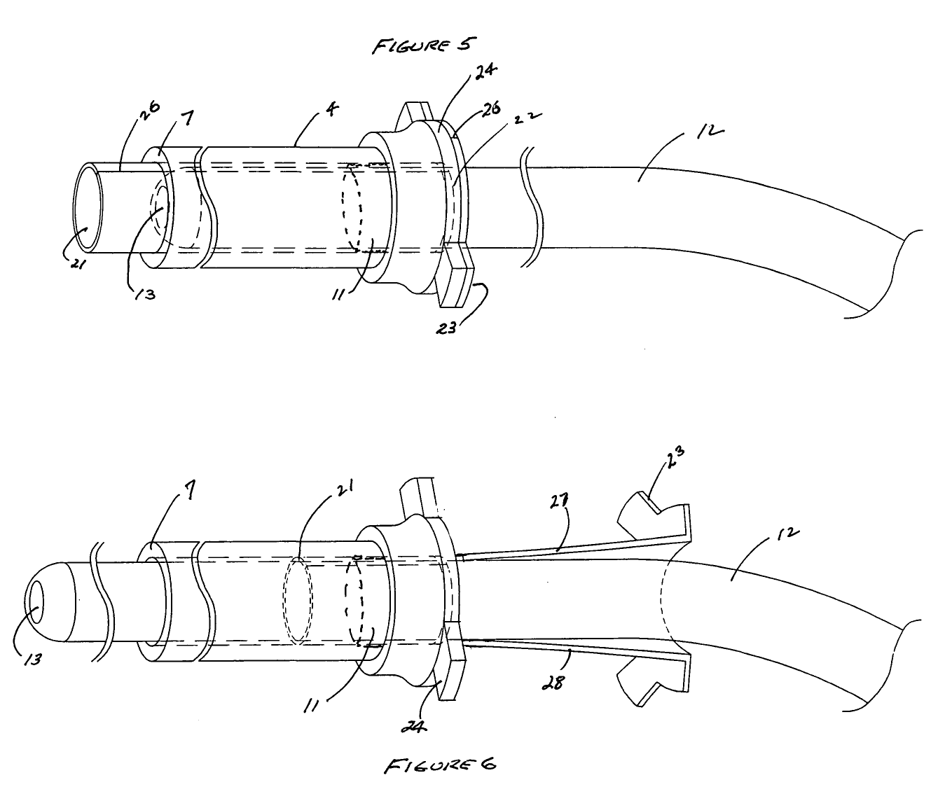

In another embodiment, shield member 14 has a slit 26 that extends axially for the entire axial length of shield member 14. As can be seen in FIGS. 5 and 6, shield member 14 is made of an elastic material that is sufficiently elastic to permit slit walls 27 and 28 to be circumferentially displaced from each other to expand the arcuate circumferential distance between walls 27 and 28 which allows shield member 14 to be laterally removable from or attachable to flexible or rigid surgical endoscope 12. Referring again to FIGS. 5 and 6, FIG. 5 illustrates the passage of lens 13 through shield lumen 19 in an anterior-posterior direction in gaining access to the abdominal region. In FIG. 5, flange 23 has engaged anterior barrier 24 which precludes further axial advance posteriorly of shield member 14 through working port lumen 3. Shield member 14 is so dimensioned and proportioned such that it may advance axially through working port lumen 3 at least a sufficient distance so that the first opening 21 of shield member 14 will be posteriorly removed from working port valve 11. With shield member 14 so positioned, lens 13 of flexible or rigid surgical endoscope 12 may be inserted into and passed through shield lumen 19 thereby by-passing working port valve 11.

While I have shown and described embodiments of a shield member for shielding the lens of a flexible or rigid surgical endoscope, it is to be understood that the invention is subject to many modifications without departing from the scope and spirit of the claims as recited herein.

What is claimed is:

1. A device for shielding the lens of a flexible or rigid surgical endoscope during passage of said lens through a working port, said working port having a working port lumen, a posterior end, an anterior portion and a working port valve, said device comprising a shield member having a distal end, a proximal end, an axis of elongation, and a void extending axially therethrough, said shield member further having a first opening at said distal end communicating with said void and a second opening at said proximal end communicating with said void where said shield member is so dimensioned and proportioned to permit said shield member to be telescopically carried within said working port lumen and to permit said first opening to be positioned posteriorly with respect to said working port valve, and where said void is so dimensioned and proportioned to permit said lens to advance axially within said void such that said lens may be posteriorly spaced relative to said working port valve.

2. The device recited in claim 1 where said anterior portion of said working port has an anterior barrier and said shield member has a radially extending flange adjacent said proximal end such that upon engagement of said radially extending flange with said anterior barrier, said first opening of said shield member is precluded from axial displacement posteriorly relative to said working port lumen.

3. The device recited in claim 1 where said shield member is made of an elastic material.

4. The device recited in claim 3 where said shield member has an axially extending slit communicating with said void where said elastic material is sufficiently elastic to permit said slit to be circumferentially expanded such that said shield member may be laterally removable through said slit from said flexible or rigid surgical endoscope.

5. The device recited in claim 2 where said shield member is made of an elastic material.

6. The device recited in claim 5 where said shield member has an axially extending slit communicating with said void where said elastic material is sufficiently elastic to permit said slit to be circumferentially expanded such that said shield member may be laterally removable through said slit from said flexible or rigid surgical endoscope.

7. The device recited in claim 1 where said void is cylindrically shaped.

8. The device recited in claim 4 where said void is cylindrically shaped.

9. In combination:
(a) a working port having a working port lumen, a posterior end, an anterior portion, and a working port valve; and
(b) a shield member having a distal end, a proximal end, an axis of elongation, and a void extending axially therethrough, said shield member further having a first opening at said distal end communicating with said void and a second opening at said proximal end communicating with said void, where said shield member is so dimensioned and proportioned to permit said shield member to be telescopically and slidably carried within said working port lumen and where said void is so dimensioned and proportioned to permit said first opening to be spaced posteriorly of said working port valve within said working port lumen.

10. The combination recited in claim 9 where said anterior portion of said working port has an anterior barrier and said shield member has a radially extending flange adjacent said proximal end such that upon engagement of said radially extending flange with said anterior barrier said first opening is precluded from further axial displacement posteriorly relative to said working port lumen.

11. The combination recited in claim 9 where said shield member is made of an elastic material.

12. The combination recited in claim 11 where said shield member has an axially extending slit communicating with said void where said elastic material is sufficiently elastic to permit said slit to be circumferentially expanded to a selectively arcuate distance.

13. The combination recited in claim 10 where said shield member is made of an elastic material.

14. The combination recited in claim 13 where said shield member has an axially extending slit communicating with said void where said elastic material is sufficiently elastic to permit said slit to be circumferentially expanded to a selectively arcuate distance.

15. The combination recited in claim 9 where said void is cylindrically shaped.

16. The combination recited in 14 where said void is cylindrically shaped.

17. The combination recited in claim 10 further comprising in combination a flexible or rigid surgical endoscope having a distal end and a lens carried by said flexible or rigid surgical endoscope adjacent said distal end.

18. The combination recited in claim 14 further comprising in combination a flexible or rigid surgical endoscope having a distal end and a lens carried by said flexible or rigid surgical endoscope adjacent said distal end.
